(19) United States
(12) Patent Application Publication
(10) Pub. No.: US 2024/0197344 A1
(43) Pub. Date: Jun. 20, 2024

(54) HOLDING DEVICE FOR A LITHOTRIPSY DEVICE, AND LITHOTRIPSY DEVICE FOR FRAGMENTING CALCULI

(71) Applicant: KARL STORZ SE & Co. KG, Tuttlingen (DE)

(72) Inventors: Bernhard GLÖGGLER, Tuttlingen (DE); Beat KRATTIGER, Tuttlingen (DE)

(21) Appl. No.: 18/535,680

(22) Filed: Dec. 11, 2023

(30) Foreign Application Priority Data
Dec. 15, 2022 (DE) 10 2022 133 521.6

(51) Int. Cl. A61B 17/22

(52) U.S. Cl. CPC A61B 17/22012; A61B 2017/22015; A61B 2017/22025

(57) ABSTRACT

The invention relates to a holding device for a lithotripsy device for fragmenting calculi, the holding device comprising a housing with a distal end and a proximal end and a sonotrode being connectable to the distal end, arranged within the housing there being an acceleration tube with a longitudinal center axis, a cavity, a proximal end, and a distal end, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth between the proximal-side abutment element and the distal-side abutment element, and a vibration excitation apparatus for exciting vibrations of the sonotrode being arranged in the housing, wherein the holding device comprises a vibration compensation apparatus with at least one mass and at least one spring element such that the vibration compensation apparatus makes it possible to decouple the acceleration tube from the excitation of vibrations by means of the vibration excitation apparatus. The invention also relates to a lithotripsy device for fragmenting calculi.

### CROSS-REFERENCE TO RELATED APPLICATIONS

This application claims the benefit under 35 U.S.C. 119(a) to German Patent Application No. 10 2022 133 521.6, filed 15 Dec. 2022, the disclosure of which is incorporated herein by reference in its entirety.

### BACKGROUND

The invention relates to a holding device for a lithotripsy device for fragmenting calculi, the holding device comprising a housing with a distal end and a proximal end and a sonotrode being connectable to the distal end, arranged within the housing there being an acceleration tube with a longitudinal center axis, a cavity, a proximal end, and a distal end, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth between the proximal-side abutment element and the distal-side abutment element, and a vibration excitation apparatus for exciting vibrations of the sonotrode being arranged in the housing. The invention also relates to a lithotripsy device, in particular an intracorporeal lithotripsy device, for fragmenting calculi.

Lithotripsy is a known method for fragmenting calculi which form, for example, through condensation and/or crystallization of salts and proteins as concretions in body organs, such as in the bladder or kidneys. If the calculi are too large to be passed naturally and cause discomfort, they have to be fragmented with a lithotripter so that the fragmented stones can be removed by natural excretion and/or by means of an aspiration/irrigation pump. The calculi to be subject to fragmentation are frequently structured inhomogeneously with different constituent parts and/or solidities.

To improve calculi fragmentation performance, use is made, especially in intracorporeal lithotripsy, of combination systems which combine two different excitation and/or vibration sources. To this end, intermittent, ballistic shockwave energy is frequently supplied in addition to the constant ultrasonic energy. For example, this can be implemented by means of a ballistic drive with electromagnets, in which an impact body is accelerated by means of the electromagnets and strikes on a horn and/or the sonotrode head. Disadvantageously, at least the distal end of the grip of such a lithotripter needs to be actively cooled in this case on account of the heating of the electromagnets during constant operation. Moreover, the securely connected hybrid cable necessary for the cooling renders the instrument heavy and ergonomically cumbersome.

Moreover, the practice of arranging an oscillating mass in ring form around a sonotrode and pressing said mass against an axial stop of the sonotrode by means of a spring is known. In this case, the ultrasonic vibration accelerates the mass away from the stop, whereby the spring is compressed and accelerates the mass back toward the stop. A disadvantage of this spring-mass system acting in the longitudinal direction of the sonotrode is that said system enables only a restricted punch. Moreover, the ballistic function cannot simply be added as desired and cannot be set in terms of its strength, repetition rate and/or cadence.

In the case of ballistic systems with a purely pneumatic drive or combination systems with a pneumatic unit, the projectile is moved in the distal direction within an acceleration tube and needs to be moved back in the proximal direction following an impact on the probe or sonotrode. To return the projectile, an air reservoir with a valve may be arranged distally or, as described in DE 20 2014 007 692 U1, a storage chamber may be arranged around the acceleration tube. A disadvantage of the two return variants is that the installation space in the pneumatic unit and/or lithotripsy device is restricted on account of the air reservoir or the storage chamber and the through-routing of other components, for example irrigation and aspiration lines, is rendered more difficult from a constructional point of view.

In the context of exciting vibrations by means of ultrasound, it is also known to arrange an ultrasonic vibration compensator in ultrasonic transducers, on the side opposite to the horn and hence at the vibrating proximal end of the ultrasonic converter, said vibration compensator serving as a mechanical fastening element between a housing of the lithotripsy device at rest and the vibrating, proximal end of the ultrasonic converter. In the case of a targeted design of this ultrasonic vibration compensator, the latter reduces the ultrasonic vibrations to a minimum or zero over its length, without the ultrasonic converter being noticeably detuned in terms of its resonant frequency in the process. However, it is not possible to design the dimensions of such an ultrasonic vibration compensator to take any desired values since, if this were the case, the ultrasonic converter would undesirably detune, unwanted transverse vibrations would be excited, and/or uncomfortable noises might occur. Moreover, it is not possible to freely design the housing length in the proximal direction.

It is an object of the invention to improve the prior art.

The object is achieved by a holding device for a lithotripsy device for fragmenting calculi, the holding device comprising a housing with a distal end and a proximal end and a sonotrode being connectable to the distal end, arranged within the housing there being an acceleration tube with a longitudinal center axis, a cavity, a proximal end, and a distal end, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth between the proximal-side abutment element and the distal-side abutment element, and a vibration excitation apparatus for exciting vibrations of the sonotrode being arranged in the housing, wherein the holding device comprises a vibration compensation apparatus with at least one mass and at least one spring element such that the vibration compensation apparatus makes it possible to decouple the acceleration tube from the excitation of vibrations by means of the vibration excitation apparatus.

Consequently, a handpiece is provided for a lithotripsy device with a combined shock and vibration excitation and a vibration compensation apparatus for the compensation of ultrasonic vibrations. Consequently, undesirably excited vibrations on the acceleration tube, which are generated by the vibration excitation apparatus, are prevented or at least reduced, whereby the vibration excitation apparatus and the ballistic drive are settable and operable independently of one another by means of the force generation apparatus. It is particularly advantageous that on account of the multifunctional vibration compensation apparatus the holding device for a combined lithotripsy device has a low weight, need not be cooled, and has a shock excitation that can be set and added as desired. Consequently the acceleration tube which is at rest, and hence stationary, is securely held in the housing of the holding device and at the same time reliably decoupled by means of the vibration compensation apparatus from the significant ultrasonic vibrations at the distal end of the ultrasonic converter.

As a result of the vibration compensation apparatus comprising at least one mass as its own and independent rest mass, the vibration compensation apparatus and the housing are designable independently of one another. In this case, the vibration compensation apparatus is exactly matched to the respective ultrasonic vibrations, for example at a frequency of approximately 27 kHz, and, on account of the free designability of the vibration compensation apparatus, the latter has a λ/4 geometry, which corresponds to the resonant frequency of the ultrasonic converter and as a result does not detune or hardly detunes the latter.

On account of the multifunctional vibration compensation apparatus, a light holding device is provided as the handpiece of a combined lithotripsy device with an optimally usable installation space, which is producible in structurally simple and cost-effective fashion.

An essential concept of the invention is based on ensuring, by means of a vibration compensation apparatus with at least one spring element and with at least one mass as its own, integrated rest mass, a free design of this vibration compensation apparatus independently of the design of the housing within the holding device and thereby simultaneously providing further advantageous functions for a combined lithotripsy device in addition to the decoupling of the vibrations of the acceleration tube of the ballistic shock excitation by means of the vibration compensation apparatus, and in the process efficiently using a compact installation space within the holding device.

The following concepts shall be explained:

A “lithotripsy device” (also called a “lithotripter”) is in particular a device for fragmenting calculi by shocks, shock waves and/or deformation waves. A lithotripsy device is understood to include, in particular, various constituent parts, constructional and/or functional components of a lithotripter. The lithotripsy device can completely or partially form a lithotripter. A lithotripsy device can be in particular an intracorporeal or extracorporeal lithotripsy device. In the case of an intracorporeal lithotripsy device, the latter can additionally comprise an irrigation/aspiration pump. The lithotripsy device can be designed as hand-held equipment and/or can comprise an endoscope or be inserted into an endoscope. The lithotripsy device is in particular autoclavable and has, for example, instrument steel and/or plastic. The lithotripsy device can comprise further components, such as control and/or supply equipment, or this is assigned to the lithotripsy device. In particular, a lithotripsy device is a combined lithotripsy device with a ballistic and/or pneumatic unit and an assignable force generation apparatus and a vibration excitation apparatus. By way of a shock energy when a projectile strikes a distal-side abutment element, a deformation wave shaped in a targeted manner is impressed directly or indirectly on the sonotrode, in particular, by means of the ballistic and/or pneumatic unit and the assigned force generation apparatus. The deformation wave causes in particular a translational movement of the sonotrode, which causes fragmentation of stones on account of the deflection. In addition to the mechanical shock, the sonotrode in the combined lithotripsy device is excited to vibrate at the same time, in particular as a longitudinal vibration and/or transverse vibration, in particular by means of a vibration excitation apparatus, for example an ultrasonic transducer. Thus, the sonotrode is designed in particular as a waveguide for the vibration waves generated by the vibration excitation apparatus and for the deformation waves of the projectile.

The term “calculi” (also referred to as “concretions”) refers in particular to all stones in a human or animal body, which are formed for example from salts and proteins by crystallization and/or condensation. The calculi can include, for example, gallstones, urinary stones, kidney stones and/or salivary stones. As a result of the sonotrode and/or hollow probe acting on the calculi, calculus cores (also referred to as drilled cores) and/or calculus fragments in particular arise.

A “holding device” (also referred to as a “handpiece”) is a grip and/or holding part of the lithotripsy device in particular. The holding device can be in particular a handle for manual and/or automated operation and/or connection of the lithotripsy device. A holding device can also be arranged at, connected to and/or guided in an automated manner at a distal end of a robot arm. In particular, the holding device comprises a housing. The holding device can also be formed from two or more parts. For example, the holding device may comprise a separate housing for a pneumatic unit and a separate housing for the vibration excitation apparatus.

The terms “distal-side” and “distal” refer to an arrangement close to the patient's body and thus remote from the user, and/or a corresponding end or portion. Accordingly, “proximal-side” or “proximal” refers to an arrangement close to the user and thus remote from the patient's body, or a corresponding end or portion.

A “sonotrode” is in particular a component which, by the action and/or introduction of mechanical vibrations, is itself set in vibration and/or resonant vibration. In particular, the sonotrode is designed as a waveguide for the vibration waves generated by the vibration excitation apparatus and for the deformation waves resulting from the impact of the projectile which is accelerated by means of the force generation apparatus. In particular, the sonotrode is directly or indirectly connected to the vibration excitation apparatus, the ultrasonic transducer, and/or the horn. For example, the sonotrode is screwed into the distal-side end of the horn. The sonotrode comprises a sonotrode head, in particular at its proximal end, for recording, transmitting, and/or focusing ultrasonic waves and a sonotrode tip, at its distal end, for directly and/or indirectly impacting on and/or contacting calculi. The sonotrode is in particular shaped in such a way that it optimally introduces the vibration waves, the ultrasonic vibration, and the deformation waves at its distal end into the body, into the region of the body to be treated, and/or directly onto the calculus to be fragmented. In the case of ultrasonic excitation, the sonotrode operates in particular in the ultrasonic range with a frequency range from 20 kHz to 90 kHz, preferably from 20 kHz to 34 kHz. In particular, the sonotrode comprises steel, titanium, aluminum, and/or carbon. In particular, a sonotrode is a probe with for example a bar-shaped, tube-shaped, and/or hose-shaped embodiment. The sonotrode can be formed in one or more pieces. The sonotrode has in particular a diameter in a range from 0.5 mm to 4.5 mm, in particular from 0.8 mm to 3.8 mm.

An “acceleration tube” is in particular an elongated hollow body whose length has a greater dimension than its diameter. In its interior, the acceleration tube has a cavity in particular, in which a projectile can move freely in the longitudinal direction. Moreover, the acceleration tube in particular comprises a proximal end and a distal end which, following the subtraction of the projectile length, spatially define the maximum acceleration path approximately. Distally and/or at its distal end portion, the acceleration tube is surrounded, in particular at least in part, by the horn and a bolt connected to or associated with the horn. In the case of a pneumatic force generation apparatus, the acceleration tube comprises at least one opening for the entrance and/or exit of a pressure medium, in particular compressed air. As material, the acceleration tube comprises a metal in particular.

An “abutment element” is in particular a desired endpoint of the movement of the projectile along the acceleration path within the cavity in the acceleration tube, at which the accelerated projectile impacts on the abutment element, is decelerated, and/or moved in the opposite direction. In particular, a distal-side abutment element is arranged at and/or in the distal end of the acceleration tube and/or within the cavity in a region of the distal portion of the acceleration tube. The distal-side abutment element transmits the shock of the projectile onto the sonotrode, in particular directly or indirectly. For example, the distal-side abutment element can be a proximal-side wall of the horn, a spring element, or a proximal-side wall of a holder for a spring element. In particular, the proximal-side abutment element is arranged at and/or in the proximal end of the acceleration tube or within the cavity in a proximal portion of the acceleration tube. For example, the proximal-side abutment element can be a wall of the housing and/or a spring element.

A “projectile” is in particular a body which is freely movable along the acceleration path within the cavity in the acceleration tube. The projectile is movable in particular back and forth between the proximal-side abutment element and the distal-side abutment element within the cavity, arranged therebetween, in the acceleration tube. In principle, the projectile can have any shape. For example, the projectile can have the shape of a bolt or a ball. The projectile has in particular hard steel and/or weak magnetic properties. For the free mobility, the projectile has in particular a slightly smaller outer diameter than the diameter of the cavity in the acceleration tube. For example, the projectile can have an outer diameter of 8 mm, in particular 6 mm, or 4 mm.

In particular, the projectile can be moved back and/or forth along the acceleration path continually, repeatedly or in a manner triggered on an individual basis by means of the force generation apparatus. Preferably, the projectile is moved back and forth in an intermittent and/or oscillating manner between the proximal-side abutment element and the distal-side abutment element.

In principle, a “force generation apparatus” can be any type of apparatus that applies a force to the projectile and thus causes a movement of the projectile. The force generation apparatus can be, for example, an apparatus which accelerates the projectile by means of a laser, a pressure medium, for example pneumatically by means of compressed air, by means of an electromagnetic field and/or by means of a mechanical apparatus. A pneumatic force generation apparatus can bring about a linear movement of the projectile in the cavity in the acceleration tube by means of a supply and/or removal of a pressure medium in particular. In particular, the pressure medium flows into the cavity in the acceleration tube through at least one proximal-side opening in the acceleration tube and presses and accelerates the projectile in the distal direction.

In particular, a “vibration excitation apparatus” is any apparatus for generating vibrations in the ultrasonic range. In particular, the vibration excitation apparatus comprises an ultrasonic transducer (also referred to as an ultrasonic converter) which converts a supplied AC voltage at a specific frequency into a mechanical vibration frequency; or the vibration excitation apparatus is formed by the ultrasonic transducer. In particular, the ultrasonic transducer is an electromechanical transducer that exploits the piezoelectric effect. As a result of applying the AC voltage generated by an ultrasonic generator, a mechanical vibration is generated on account of a deformation of the ultrasonic transducer. In particular, the ultrasonic transducer comprises a piezo element or a plurality of preferably stacked piezo elements. Preferably, the ultrasonic transducer comprises at least two piezo elements, with an electrical conductor, for example a copper plate, being arranged between the piezo elements. A distal-side piezo element of the ultrasonic transducer rests, especially directly, against a proximal wall of a horn. In particular, a counter bearing is arranged to the proximal side of the piezo element or the piezo elements. An intermediate plate can be arranged between the proximal end of the proximal-side piezo element and the distal end of the counter bearing. The piezo element, the piezo elements, the intermediate plate, and/or the counter bearing can be arranged in particular around a bolt, in particular a hollow bolt, which is arranged to the proximal side of the horn.

In particular, a “horn” is a component arranged between the ultrasonic transducer and/or a piezo element on the one hand and the sonotrode on the other hand. In particular, the horn serves to transfer the ultrasonic waves generated by the ultrasonic transducer to the sonotrode, and/or to transmit, to focus, and/or to align said ultrasonic waves. To this end, the horn may taper in a transfer direction and directly or indirectly transfer the ultrasonic waves to a probe head. In particular, an amplitude increase is obtained as a result of a cross-sectional reduction of the horn in the transfer direction. The horn can also be used for fastening the sonotrode. At the same time, the horn serves in particular together with a counter bearing and/or an intermediate plate for mechanically holding the piezo element or piezo elements on both sides. The horn terminates with a wall counter to the transfer direction, in particular on the proximal side. A bolt in particular is arranged to the proximal side of this wall. The bolt is preferably a hollow bolt. In particular, the horn and the bolt can be designed as two separate components. Preferably, the horn and the bolt are a one-piece component, with the horn portion corresponding to the conventional horn and merging into the hollow bolt portion with a smaller cross section, especially in graduated fashion, counter to the transfer direction, in particular in the proximal direction. At least one piezo element with an electrical contact and the counter bearing and/or additionally an intermediate plate between the proximal-side piezo element and the distal side of the counter bearing arranged therebetween are arranged around the hollow bolt portion. In particular, the counter bearing is screwed onto the hollow bolt or the hollow bolt portion and as a result clamps at least one piezo element and/or the intermediate plate. The counter bearing can be designed as a screw nut. A proximal end portion of the hollow bolt portion and/or hollow bolt protrudes beyond the proximal end of the counter bearing, especially in the proximal direction. In particular, a connection portion of the vibration compensation apparatus is arranged and/or connected in circumferential fashion by way of this protruding proximal end portion of the hollow bolt portion and/or hollow bolt. Preferably, the connection portion of the vibration compensation apparatus is screwed onto the proximal portion of the hollow bolt portion and/or hollow bolt and consequently mechanically coupled to the latter. As a result, the proximal end of the ultrasonic transducer in particular is mechanically coupled to the connection portion of the vibration compensation apparatus.

In particular, a “vibration compensation apparatus” (also referred to as “amplitude compensator”) is a component or an assembly comprising at least one mass and at least one spring element. In particular, the vibration compensation apparatus serves to vibrationally decouple the acceleration tube of the ballistic and/or pneumatic drive from the vibration excitation by means of the vibration excitation apparatus. In particular, the spring element is arranged on the distal side and the mass, as a rest mass, is arranged on the proximal side of the vibration compensation apparatus. The vibration compensation apparatus comprises in particular a continuous cavity in its mass and its spring element, through which the acceleration tube can be guided, with the result that the outer surface of the acceleration tube is surrounded by the vibration compensation apparatus.

As further components, the vibration compensation apparatus comprises, in particular, at least one connection element for connecting the mass and at least one sealing element such as an O-ring, for example. The sealing element acts as a damping element at the same time. The vibration compensation apparatus may also comprise a plurality of spring elements, for example arranged parallel to one another, and/or a plurality of masses.

In particular, a “spring element” is a component and/or a portion of the vibration compensation apparatus that can be elastically deformed to a sufficient extent. In particular, the spring element comprises metal and/or plastic. In particular, a spring element can be a conventional spring, for example a coil spring, and hence a wire wound in a coil. Preferably, the spring element is a thin-walled tube portion which in particular acts as a λ/4 mass spring element. The mass and/or the entire vibration compensation apparatus comprises aluminum and/or steel in particular. Preferably, the entire amplitude compensator comprises aluminum and/or an aluminum alloy. While the spring element of the vibration compensation apparatus vibrates during operation and thus has a damping effect, the mass remains at rest in particular on account of its significantly higher weight and precisely does not vibrate.

An “amplitude node” is a zero-deflection location in the region of a standing wave or of the superposition of two opposing traveling waves at the same frequency and with same amplitude, which has arisen from reflection. The “wavelength” of a periodic wave is the smallest distance between two points of the same phase in particular. For example, the wavelength is the distance between two maximal amplitudes.

A “λ/4 geometry” of the vibration compensation apparatus is understood to mean in particular that, when the ultrasonic transducer is fastened to an amplitude antinode (vibration antinode), the spring element of the vibration compensation apparatus has a distance of λ/4 from the proximal mass of the vibration compensation apparatus, which is to say to the amplitude node corresponding thereto. As a result, the maximum amplitude is present at the distal end of the spring element and this is damped in the proximal direction on account of the elastic properties of the spring element, with the result that there is only a small or no residual ultrasound amplitude present in the mass of the vibration compensation apparatus arranged to the proximal side, whereby vibrational decoupling from the acceleration tube at rest is obtained. As a result of the vibration compensation apparatus having a λ/4 geometry, the latter corresponds to the resonant frequency of the ultrasonic transducer and does not detune the ultrasonic transducer or only detunes the latter very slightly.

A “longitudinal center axis” is in particular the axis of the respective body or component which corresponds to the direction of its greatest extent and/or dimension. The longitudinal center axis can also be the axis of symmetry of the respective body and/or component.

In particular, a “longitudinal direction” is the direction of the longest extent of a component and/or body. In particular, the longitudinal direction is the direction along the longitudinal center axis of the mass, sonotrode, and/or acceleration tube.

In its longitudinal direction and/or at its proximal end, the mass is arranged without a connection to the housing in a further embodiment of the holding device.

Consequently, the mass of the vibration compensation apparatus is in the form of a rest mass which is free in the longitudinal direction and precisely not a part of the housing or connected to the housing on the proximal side. As a result, further functions can be integrated freely into the vibration compensation apparatus. In particular, the installation length of the housing and/or holding device, especially in the proximal direction, is independent of the ultrasonic function of the ultrasonic transducer. As a result, the installation length of the housing can be customized to the length of the acceleration tube or can be chosen freely. By virtue of the vibration compensation apparatus being designed independently of the housing back wall since the mass in the vibration compensation apparatus is integrated as a rest mass which is independent of the housing back wall, the ballistic and/or pneumatic properties, and hence the shock excitation, can be optimized and set independently of the ultrasonic properties and hence independently of the vibration excitation apparatus. As a result, the acceleration tube can be optimally installed in the housing, even if the latter must usually have a specific length for efficient functioning. As a result of the decoupling by means of the vibration compensation apparatus and the rest mass integrated in the vibration compensation apparatus itself, the housing of the holding device can be lengthened as desired, especially in the proximal direction, without the ultrasonic conditions of the vibration excitation apparatus changing in the process.

To dissipate transverse moments of the vibrations generated by means of the vibration excitation apparatus, the mass is directly or indirectly connected to the housing substantially transversely to the longitudinal direction of said mass by means of at least one connection element.

As a result, transverse moments on the ultrasonic converter mounted elastically and movably on the node of the horn are intercepted by means of the vibration compensation apparatus and dissipated in a targeted fashion substantially in a direction transversely to the longitudinal direction of the mass and/or acceleration tube. Hence, the amplitude compensator can be screwed to the vibrating proximal end of the ultrasonic transducer and/or the hollow bolt while the thicker opposite component end of the amplitude compensator, as a rest mass, is directly or indirectly connected to the housing, and hence mounted, by means of at least one connection element via the lateral surface of said amplitude compensator.

In particular, “substantially transversely to the longitudinal direction” is understood to mean that the connection between the mass of the amplitude compensator and its longitudinal direction need not necessarily have an angle of 90°. Thus, the longitudinal axis of the connection element may also include an angle smaller than 90° with the longitudinal direction of the mass and/or acceleration tube; for example, this angle can be 60°.

Moreover, the fastening of the mass of the amplitude compensator by means of at least one connection element in addition to the conventional fastening of the ultrasonic transducer to its vibration nodes in the longitudinal direction leads to the ultrasonic transducer being connected more stably to the housing, thus allowing greater forces and moments to be transferred.

In a further embodiment of the holding device, the mass is directly or indirectly connected to the housing by means of at least three radially uniformly spaced apart connection elements.

As a result, the mass of the amplitude compensator is mounted to the surrounding housing in radially circumferential fashion by means of at least three uniformly distributed connection elements and the vibrations and/or forces are dissipated into the housing via the lateral surface of the mass in uniform or non-uniform fashion, depending on the moment.

As a result of the at least one connection element or the at least three radially uniformly spaced apart connection elements and, optionally, an additional sealing element, for instance an O-ring as a damping element, being arranged in and/or on the mass as a rest mass, these have a negligibly small movement on account of the small residual ultrasound amplitude and consequently an abrasion, a loss, and/or a heating of the at least one connection element, the connection elements, and/or a sealing element is very small and/or negligible.

To be able to compensate torques which may arise on account of a linear mount of the horn, a respective punctiform direct or indirect connection to the housing is formed by means of the at least one connection element or the connection elements.

The punctiform connection or the punctiform connections lead to the interception of transverse moments due to which the proximal end of the ultrasonic converter would otherwise collide with the housing wall, and this may lead to noises, faults, and damage in addition to impairments of the function. Moreover, without this transfer of moments by means of the punctiform connection of the respective connection element to the housing or the indirect connection to the housing via another component, the soft, resilient mount of the housing would cause a wobbly, imprecise, and hence disadvantageous sensation for the user when handling the holding device and/or lithotripsy device. Moreover, detuning of the ultrasonic converter and too great a loss of the vibration power in the housing are prevented by the punctiform mount of the mass of the amplitude compensator.

In a further embodiment of the holding device, the at least one connection element or the connection elements comprises or comprise plastic.

Residual ultrasound amplitudes do not lead to metallic rattling as a result of the at least one connection element or the connection elements comprising plastic and hence a plastic surface being present in the case of the connection in contact with the housing or the indirect connection to the component of the housing. Consequently, the housing or a component in the housing, against which the connection element rests or the connection element rest, may comprise metal.

In particular, a “connection element” is an element which establishes a mechanical connection between the mass of the vibration compensation apparatus and the housing or a component in the housing. In particular, the connection element may establish an interlocking and/or frictionally connected connection. Likewise, the mass can be connected loosely and guided with a little play on and/or in a component in the housing or the housing by means of the connection element or the connection elements. For example, the connection element is a pin or bolt. The connection element preferably comprises plastic. The connection element can be formed specifically in order to establish a punctiform connection and hence a connection only at one point or a small area of the connection element. To this end, the connection element may comprise a tip or a detent for example, which for example engages in a cutout or a fluting in the housing or in a component within the housing. Consequently, each connection element preferably comprises a specifically shaped small-area bearing point made of plastic. At the same time, additional damping is realized by the plastics material of the connection element. The connection element can also be a bolt which comprises plastic and a spherical contact face. The transverse moments are aligned optimally on a contact point and dissipated by the latter by way of the spherical geometry or a spherical or hemispherical end of the connection element. In order to easily produce a spring element and arrange the latter around the acceleration tube and/or the hollow bolt to the proximal side of the horn, the at least one spring element is in the form of a tube portion, with a wall thickness of the tube portion being smaller than a material thickness of the mass.

As a result of the tube portion having a significantly thinner wall than the material thickness of the mass of the vibration compensation apparatus, the tube portion acts directly as λ/4 mass spring element. It is particularly advantageous that the amplitude compensator is able to be fabricated as a one-piece component with the distal-side tube portion and the proximal-side mass.

In particular, the “material thickness” of the mass is the material thickness of the mass from its lateral surface to its inner surface adjacent to the acceleration tube. In particular, the “wall thickness” is the thickness of the tube of the tube portion.

In a further embodiment of the holding device, the vibration compensation apparatus comprises a cavity and/or a cutout for accommodating a pressure medium, and optionally at least one sealing element.

As a result, a further function of the vibration compensation apparatus is provided by virtue of the cutout and/or a cavity in the vibration compensation apparatus providing a reservoir or chamber in which, in the case of a pneumatic force generation apparatus, the pressure medium is compressed when the projectile is moved in the distal direction and, following the impact of the projectile on the distal-side abutment element, the compressed pressure medium can be used for the return movement of the projectile in the opposite, proximal direction. As a result, a compressed air spring for returning the projectile can be realized by virtue of a counter pressure being built up in the cutout and/or the cavity in the vibration compensator by means of the compressed pressure medium, in particular compressed air, with the result that, when a pneumatic valve is switched off under venting conditions, the projectile can be reliably moved back into its initial position again by means of the compressed air. Consequently, the amplitude compensator represents a combination component which provides both vibration decoupling and an interior volume for accommodating the compressed pressure medium. To this end, the cavity and/or the cutout in the vibration compensation apparatus is set accordingly in terms of its size and hence accommodation volume, in order to prevent the building-up counter pressure, as a compressed air spring, attenuating the impact of the projectile too strongly in the case of a volume that is too small, and hence reducing the fragmentation performance. Then again, it is not possible to increase the volume to any desired size as this would otherwise lead to a compression of the compressed air in the volume that is too small and the projectile accordingly experiencing a return spring pressure that is too low. In principle, the cavity and/or the cutout can be arranged freely within the vibration compensation apparatus. Preferably, the cavity and/or the cutout is formed at least in part in the tube portion. In the case of a cutout, the latter is for example introduced into the inner wall of the tube portion such that the volume for accommodating the pressure medium is arranged between the inner wall of the tube portion at the cutout and the outer surface of the acceleration tube. In particular, this volume ranges from 3 ml to 16 ml, preferably from 5 ml to 11 ml. If a projectile with an external diameter of 8 mm is used, the volume for accommodating the pressure medium can be 7.6 ml in particular. The usable volume formed by means of the cavity and/or the cutout for compressed air (air reservoir) is designed in particular for a high projectile frequency and/or shock frequency. If the volume is designed as a cutout in for example the inner surface of the tube portion of the vibration compensation apparatus, this volume is sealed by means of a sealing element, for example an O-ring, or a plurality of sealing elements. In particular, the acceleration tube and/or the cutout, as a compressed air chamber, is sealed from the interior of the housing around the ultrasonic transducer. Preferably, this air reservoir for returning the projectile is sealed vis-à-vis the acceleration tube using a proximal sealing element and sealed between the connection portion of the amplitude compensator and the hollow bolt portion and/or hollow bolt proximally of the horn using a distal sealing element, whereby the compressed air cannot flow into the interior of the housing. Since the tube portion is not sealed from the acceleration tube on the distal side, the compressed air can flow in the proximal direction or in the distal direction between the air reservoir and the distal end of the acceleration tube through a compressed air channel between the inner surface of the tube portion, the hollow bolt portion, and the horn and the outer surface of the acceleration tube.

Moreover, the proximal-side sealing element prevents rattling since it prevents a metallic contact between the acceleration tube and the rest mass of the amplitude compensator subject to a residual amplitude. In addition to the sealing function, the distal-side and proximal-side sealing elements of the amplitude compensator absorb additional vibrations.

To optimally exploit the installation space within the housing and obtain efficient decoupling, the vibration compensation apparatus is at least partially arranged around the acceleration tube.

In a further embodiment of the holding device, the vibration compensation apparatus is arranged concentrically around the acceleration tube.

As a result, a uniform, radially circumferential decoupling of the acceleration tube from the vibration excitation apparatus is obtained.

In order to establish an indirect connection between the mass of the amplitude compensator and the housing and optimally exploit the available installation space, the holding device comprises a circuit board holder, the circuit board holder being at least partially arranged around the vibration compensation apparatus and the mass of the vibration compensation apparatus being connected to the circuit board holder by means of the at least one connection element.

Hence, the circuit board holder has the dual function as a carrier element for electronic components within the holding device and as a mount for the mass of the amplitude compensator, and hence as an indirect connection component for mounting the mass by means of a connection element or a plurality of connection elements.

In a further embodiment, the holding device comprises a horn distally and a bolt proximally of the horn, the horn and the bolt surrounding a distal portion of the acceleration tube, a counter bearing is arranged on the bolt proximally of the horn and at least one piezo element as a vibration exciter is arranged and mechanically coupled between the counter bearing and the horn, the horn comprising the distal-side abutment element and/or the horn being connectable to the distal-side abutment element and/or sonotrode and the at least one piezo element being electrically connectable to an assignable ultrasonic generator, the vibration compensation apparatus being arranged proximally on and/or of the horn, the bolt, and/or the counter bearing.

To connect the amplitude compensator distally, the at least one spring element comprises a connection portion, the connection portion surrounding a proximal end portion of the bolt and/or being arranged proximally of the counter bearing.

As a result, the amplitude compensator can be screwed onto the vibrating proximal end of the ultrasonic transducer and/or bolt by means of the connection portion. Consequently, a detachable interlocking and frictionally connected screw-in connection is made possible.

In a further embodiment of the holding device, the mass comprises a perforation and/or, on its outer surface, at least one cutout in its longitudinal direction for guiding a line and/or a tube.

Consequently, a further function of the amplitude compensator is realized by virtue of the latter enabling a lead-through and hence an efficient exploitation of the installation space available within the housing. As a result, a structure for guiding lines and/or tubes in the longitudinal direction of the housing is facilitated since these are guided and/or sealed to and/or through the mass with a negligibly small residual ultrasound amplitude. In this case, the cutout or a perforation through the mass provides sufficient space in the longitudinal direction for leading-through tubes and/or electrical lines, for example the electrical lines from the electrical contacts of the piezo elements to the proximal cable lead-through and/or to the socket at the proximal end of the housing. For example, the cutout can be a milled groove in the outer surface and consequently in the lateral surface of the mass along the entire longitudinal direction. For example, three uniformly spaced apart semicircular milled grooves with a large radius can be introduced into the outer surface of the mass. Likewise, this may also relate to a perforation in the longitudinal direction in an outer material region of the mass, for example correspondingly spaced apart slots distributed over the cross section of the mass.

In a further aspect of the invention, the object is achieved by a lithotripsy device, in particular an intracorporeal lithotripsy device, for fragmenting calculi, wherein the lithotripsy device comprises a sonotrode and a holding device, and the holding device is a holding device as described above.

Thus, a lithotripsy device with a handpiece is provided, which on account of the multifunctional amplitude compensator is optimally designed in respect of the efficient exploitation of the installation space, the targeted handling by a user, the freely customizable installation length of the housing, and the independent settability of the ballistic and/or pneumatic shock excitation and the ultrasonic excitation on account of the vibration decoupling.

The drawings, the description, and the claims contain numerous features in combination. It will be appreciated that the features mentioned above and the features yet to be explained below are applicable not only in the respectively specified combination but also in other combinations or on their own, without departing from the scope of the present invention.

### BRIEF DESCRIPTION OF THE DRAWINGS

The invention is explained in more detail using exemplary embodiments. In the drawing:

FIG. 1 shows a schematic three-dimensional representation of a lithotripsy device with a handpiece, a horn, and a sonotrode,

FIG. 2 shows a schematic three-dimensional representation of the handpiece with a circuit board holder around an amplitude compensator and an acceleration tube in partial section,

FIG. 3 shows a schematic three-dimensional representation of the handpiece with the amplitude compensator, the horn, and the acceleration tube in partial section,

FIG. 4 shows a schematic representation of the handpiece from FIG. 3 in full section,

FIG. 5 shows a schematic three-dimensional representation of the amplitude compensator, and

FIG. 6 shows a schematic representation of the amplitude compensator from FIG. 5 in full section.

### DETAILED DESCRIPTION

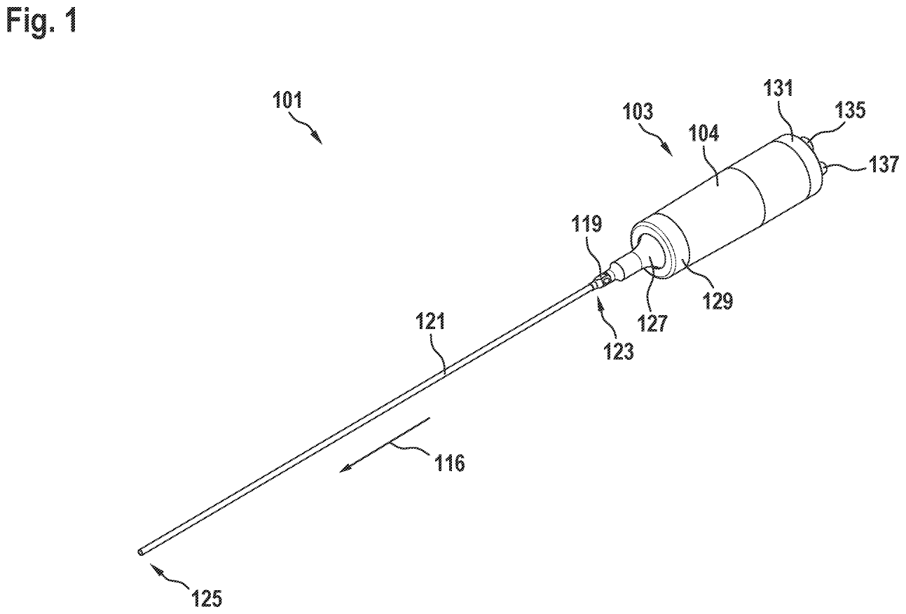

A lithotripsy device 101 comprises a handpiece 103 with a housing 104. At its proximal end, the housing 104 is terminated by a lid 131. An electrical connector 135 and a connection nozzle 137 for supplying compressed air are arranged on the proximal side of the lid 131. On the distal side, the housing 104 comprises a sleeve 129 which surrounds a horn 127. At its proximal end 123, a sonotrode 121 is screwed-in in the horn 127 by means of its sonotrode head 119. A distal end 125 of the sonotrode opposite to the proximal end 123 serves for fragmenting calculi (FIG. 1).

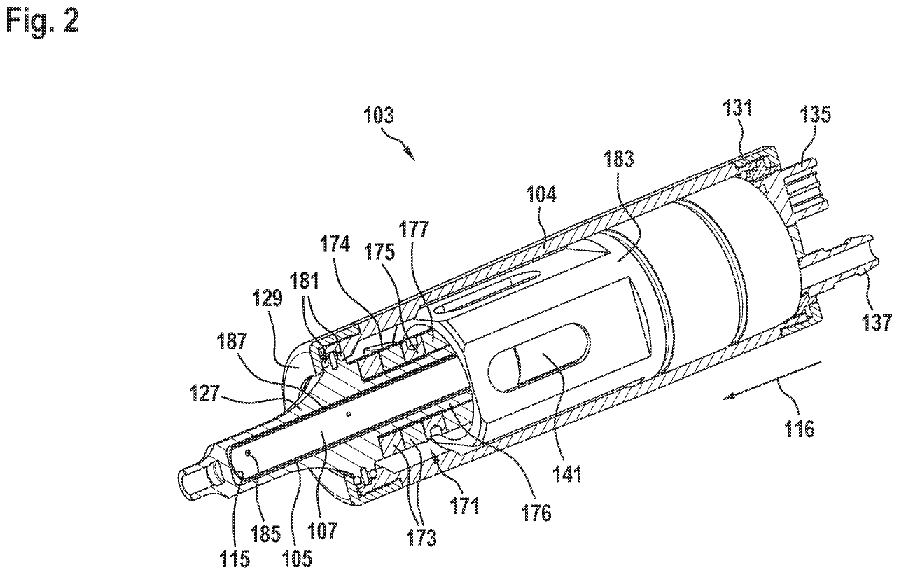

In a distal direction 116, the horn 127 has a tapering portion. To the proximal side of this tapering portion, the horn 127 merges into a hollow bolt 176 in one piece. The horn 127 is mounted in the housing 104 by means of two O-rings 181 at its largest cross section. An acceleration tube 105 which extends from its distal end 110 to its proximal end 109 along a longitudinal center axis 117 is arranged in the interior of the hollow horn 127 and the adjacent hollow bolt 176 (see FIGS. 2, 3, and 4). In the interior, the acceleration tube 105 comprises a cavity 107, in which a projectile 111 is movably arranged. The proximal end 109 of the acceleration tube 105 is held in a tube receptacle 133 within the housing 104. The cavity 107 of the acceleration tube is fluid-connected to the connection nozzle 137. The projectile 111 is movable along the longitudinal center axis 117 in the cavity 107 of the acceleration tube 105, between a proximal-side abutment element 113 and a distal-side abutment element 115. The distal-side abutment element 115 is formed by a proximal-side wall of the horn 127.

To the proximal side of the horn 127, an ultrasonic transducer 171 is arranged around the hollow bolt 176. The ultrasonic transducer 171 comprises two piezo elements 173 with an electrical conductor arranged therebetween and an electrical contact 174. The piezo elements 173 are clamped between the horn 127 and an intermediate plate 175 by means of a proximal-side counter bearing 177, with the intermediate plate 175 and the counter bearing 177 likewise surrounding the hollow bolt 176. On its outer surface, the intermediate plate 175 has holes in which a sickle spanner is placed while the piezo elements 173 are assembled on the hollow bolt 176 in order to dissipate torque during the assembly and keep this away from the piezo elements 173 as there otherwise is the risk of the piezo elements 173 twisting and becoming damaged as a result.

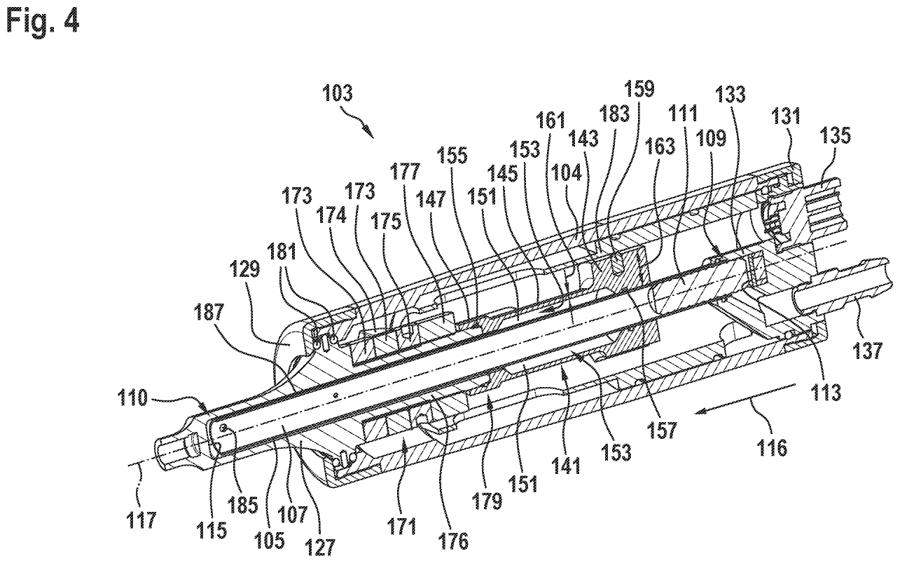

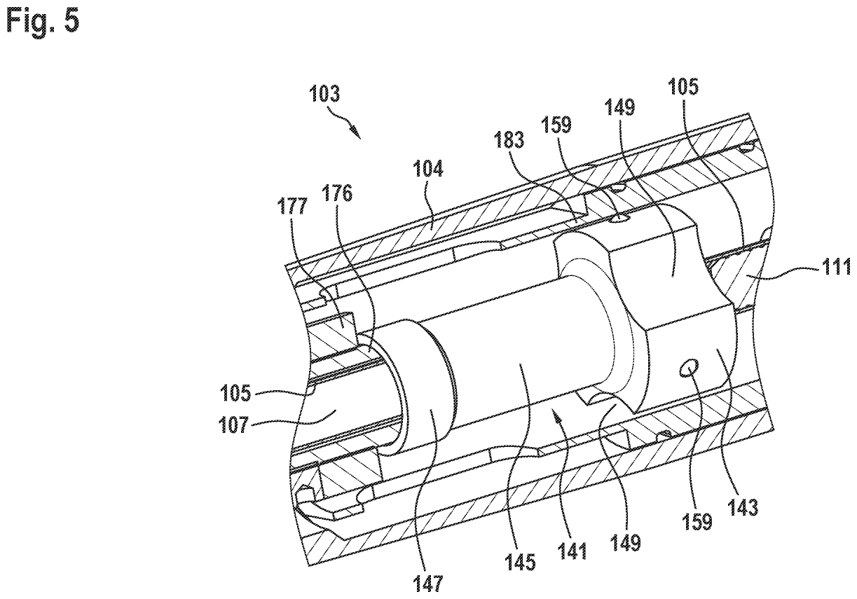

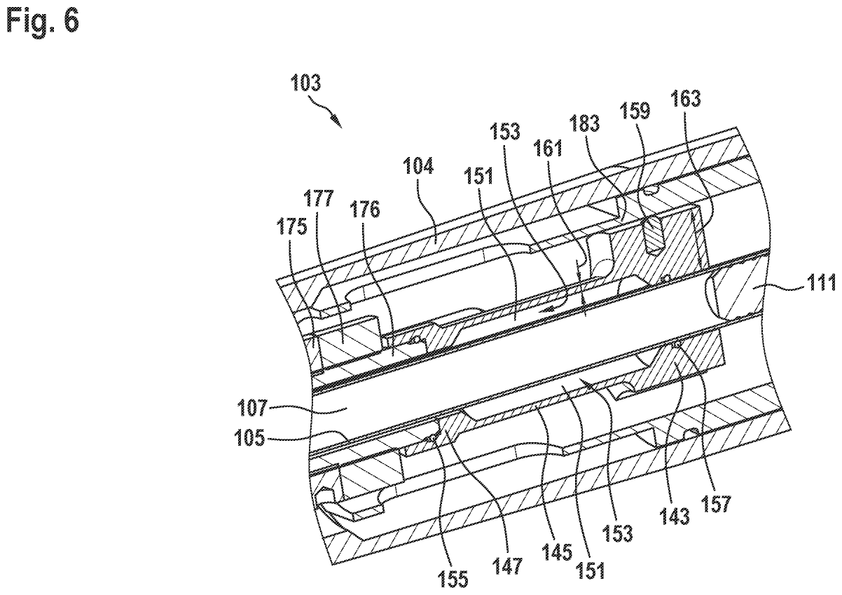

An amplitude compensator 141 is arranged around the acceleration tube 105 at the proximal end 179 of the ultrasonic transducer 171 and in the central region of the housing 104. The amplitude compensator 141 is fabricated in one piece from aluminum and has a mass part 143 on the proximal side and a spring tube portion 145 on the distal side. The spring tube portion 145 has a connection portion 147 at its distal end (FIGS. 5 and 6). The connection portion 147 is screwed onto the proximal end of the hollow bolt 176 and sealed by means of an interior distal O-ring 155. On the inside, the amplitude compensator 141 has a cavity through which the acceleration tube 105 is guided. Additionally, the amplitude compensator 141 has a cutout 151 in its inner wall around the cavity, said cutout having been introduced into the spring tube portion 145 and a distal portion of the mass part 143 such that the amplitude compensator 141 has a compressed air reservoir 153 circumferentially around the acceleration tube 105 (FIG. 4).

The mass part 143 is sealed by way of a proximal O-ring 157 at the acceleration tube 105. As a result of the amplitude compensator 141 only being sealed at the acceleration tube 105 to the proximal side by way of the proximal O-ring 157, the compressed air in the compressed air reservoir 153 formed by the cutout 151 can escape distally from the compressed air reservoir 153 through a compressed air channel 187 between the outer surface of the acceleration tube 105 and the inner surface of the distal portion of the amplitude compensator 141, hollow bolt 176, and horn 127 in the distal direction 117 and flow into the cavity 107 through an opening 185 at the distal end 110 of the acceleration tube 105 and/or through the open end face at the distal end 110 of the acceleration tube 105. Likewise, conversely, compressed air from the cavity 107 can be pressed into the compressed air channel 187 as intermediate space between the outer surface of the acceleration tube 105 and the inner surface of the horn 127 and hollow bolt 176 of the distal portion of the amplitude compensator 141 through the opening 185 and the open end face at the distal end 110 of the acceleration tube 105, pressed into the compressed air reservoir 153 counter to the distal direction 116 and collected in said compressed air reservoir when the projectile 111 is accelerated in the distal direction 116. In this case, the distal O-ring 155 between the connection portion 147 of the spring tube portion 145 and the proximal end of the hollow bolt 176 seals the compressed air channel 187 from the interior of the housing 104.

In the region of the cutout 151, the spring tube portion 145 of the amplitude compensator 141 has a significantly thinner wall thickness 161 than a material thickness 163 of the mass part 143 between the inner surface of a circuit board holder 183 and the outer surface of the acceleration tube 105. As a result of the significantly thinner wall thickness 161 of 1 mm in comparison with the 27 mm material thickness 163 of the mass part 143, the spring tube portion 145 has elastic spring properties.

In the distal direction 116, the circuit board holder 183 surrounds the acceleration tube 105 from its proximal end 109 up to and including the amplitude compensator 141 and counter holder 177. At its lateral surface, the mass part 143 of the amplitude compensator 141 is fastened in frictionally connected and interlocking fashion or guided at points on the inner surface of the circuit board holder 183 by means of three radially uniformly spaced apart plastic pins 159. In turn, the circuit board holder 183 is in contact with the inner side of the housing 104 in radially circumferential fashion, with the result that the amplitude compensator 141 is indirectly connected to the housing 104 in the radial direction via the circuit board holder 183. As a result, the proximal end of the mass part 143 is precisely without a connection to the housing 104 and the lid 131 in the proximal direction.

Moreover, the mass part 143 has three partly circular lead-through cutouts 149 that are continuous in the distal direction 116, for guiding through electrical lines (not shown in the figures) from the electrical connector 135 to the ultrasonic transducer 171.

The following operations are performed by means of the combined lithotripsy device 101 with a vibration excitation of the sonotrode 121 by means of the ultrasonic transducer 171 and a pneumatic drive for shock excitation of the sonotrode 121 by means of the projectile 111.

An ultrasonic generator (not shown in the figures) is used to apply a voltage to the ultrasonic transducer 171 by way of the electrical contact 174, whereby the piezo elements 173 are deformed within the ultrasonic transducer 171 and an ultrasonic vibration is induced as a result. The generated ultrasonic vibration is introduced into the sonotrode 121 on account of the conic portion of the horn 127, whereby the sonotrode 121 is excited to provide a vibration wave with a longitudinal vibration and in the transverse direction.

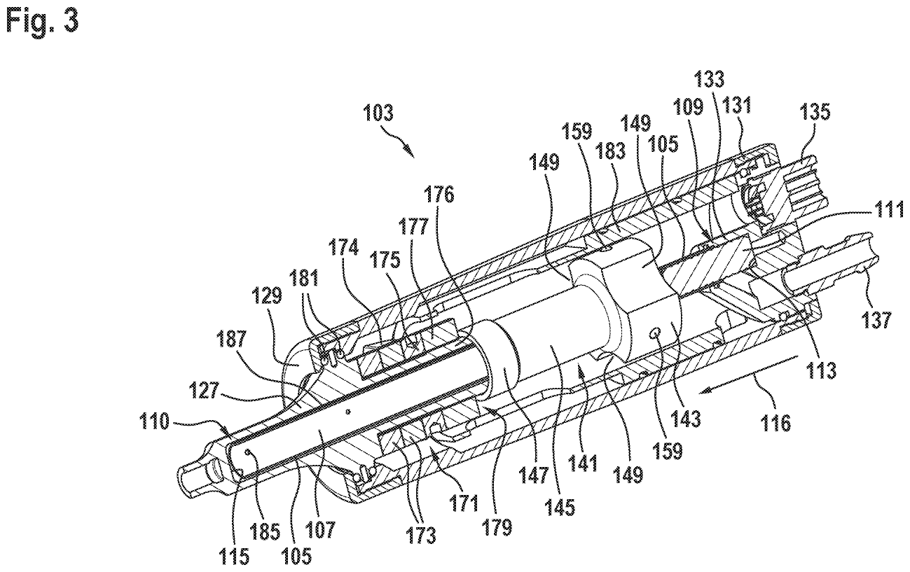

At the same time, a force generation apparatus (not shown) is used to press compressed air through the connection nozzle 137 into the cavity 107 at the proximal end 109 of the acceleration tube 105, whereby the projectile 111 moves along the longitudinal center axis 117 through the cavity 107 from the proximal end 109 as the initial state (see FIGS. 3 and 4), in the distal direction 116 from the proximal-side abutment element 113 to the distal-side abutment element 115 and, as a result of impacting on the distal-side abutment element 115, the shock from the projectile 111 is transferred to the sonotrode 121 via the distal end of the horn 127 and the sonotrode head 119. As a result of the acceleration of the projectile 111 in the distal direction 116, the air in the distal portion of the cavity 107 within the acceleration tube 105 is compressed and escapes counter to the distal direction 116 into the compressed air reservoir 153 of the amplitude compensator 141 through the opening 185 and the compressed air channel 187 between the outer surface of the acceleration tube 105 and the inner surface of the horn 127, hollow bolt 176, and distal portion of the amplitude compensator 141, with the compressed air being compressed in the compressed air reservoir 153. As a result of the impact of the projectile 111 on the distal-side abutment element 115, the projectile 111 is repulsed and, as a result of simultaneous closure of the supplied compressed air and venting through the connection nozzle 137, the compressed air compressed in the compressed air reservoir 153 now flows in the distal direction 116 through the compressed air channel 187, the opening 185, and the open distal end face of the acceleration tube 105 into the cavity 107 and presses the projectile 111 back again toward the proximal end 109 of the acceleration tube 105, until the initial state (FIGS. 3 and 4) has been reached again. This shock excitation of the sonotrode 121 as a result of the impact of the projectile 111 on the distal-side abutment element 115 is repeated on a regular basis.

The ultrasonic vibrations generated by means of the ultrasonic transducer 171 have a frequency of approximately 27 kHz, to which the amplitude compensator 141 is matched exactly. As a result of the coupled amplitude compensator 141 having a λ/4 geometry which corresponds to the resonant frequency of the ultrasonic transducer 171, the amplitude of the vibration wave generated by means of the ultrasonic transducer 171 decays continuously in the proximal direction along the spring tube portion 145 to virtually zero in the proximal mass part 143, and the ultrasonic transducer 171 is not detuned or hardly detuned by the amplitude compensator 141. In this case, the mass part 143, as a rest mass, only moves to a negligibly small extent, if at all, on account of the small residual ultrasound amplitude. In this case, the radially circumferentially arranged plastic pins 159 for a punctiform mount and the proximal O-ring 157 have an additional damping action, with the result that an abrasion, other types of damage, and heating in the mass part 143 are negligible. Moreover, metallic rattling at the circuit board holder 183 is prevented by the punctiform mount by means of the plastic pins 159, by means of which possibly present transverse moments are dissipated radially to the outside.

In addition to by way of the plastic pins 159, rattling is also prevented by the proximal O-ring 157 of the amplitude compensator 141 since this prevents metallic contact between the acceleration tube 105 and the mass part 143, as a rest mass, under residual amplitude.

As a result of the amplitude compensator 141, at its lateral surface, being mounted radially to the outside on the circuit board holder 183 by means of the plastic pins 159 and the proximal end of the mass part 143 being free in the proximal direction and precisely not connected to the housing 104 and the lid 131, the acceleration tube 105 has a length that is optimally matched to the shock effect, with the result that the pneumatic drive of the projectile 111 in the acceleration tube 105 is operable independently of the ultrasonic vibration generated by means of the ultrasonic transducer 171 and both the drives are settable independently of one another.

Consequently, when the sonotrode 121 is used for direct fragmentation of calculi, both the vibration excitation of the sonotrode 121 by means of the ultrasonic transducer 171 and the shock excitation by the projectile 111 are usable with an effective high fragmentation performance.

Moreover, the amplitude compensator 141 compensates torques, which may occur on account of a resilient linear mount of the horn 127 by means of the two O-rings 181, as a result of being mounted, radially to the outside and in a punctiform fashion, on the circuit board holder 183 and moreover on the housing 104 by means of the plastic pins 159. This interception of possible transverse moments prevents the proximal end of the ultrasonic transducer 171 from colliding with the inner wall of the housing 104, and corresponding noises, faults, and/or damage are consequently prevented. Moreover, precise handling of the housing 104 and hence precise guidance of the entire lithotripsy device 101 for the user of the combined lithotripsy device 101 are rendered possible as a result of the punctiform mount by means of the plastic pins 159.

As a result of the amplitude compensator 141 moreover providing the compressed air reservoir 153, the function of returning the projectile 111 is integrated in the amplitude compensator 141 at the same time, whereby a fast return of the projectile 111 and hence a high shock frequency is rendered possible in the case of little installation space. In particular, this renders an additional compressed air inlet to the distal side of the projectile 111 and a corresponding valve switchover for the projectile return, which are complicated and require much space, unnecessary.

Thus, a combined lithotripsy device 101 with a multifunctional amplitude compensator 141 is provided, which decouples the acceleration tube 105 from the significant ultrasonic vibration of the ultrasonic transducer 171, provides a compressed air reservoir 153 for returning the projectile 111, intercepts transverse moments, and dissipates the latter radially to the outside in a targeted manner by way of a punctiform mount by means of plastic pins 159, whereby a proximal-side length of the housing 104 is able to be designed freely and independently.

The drawings, the description, and the claims contain numerous features in combination. It will be appreciated that the features mentioned above are applicable not only in the respectively specified combination but also in other combinations or on their own, without departing from the scope of the present invention. The invention relates to a holding device for a lithotripsy device for fragmenting calculi, the holding device comprising a housing with a distal end and a proximal end and a sonotrode being connectable to the distal end, arranged within the housing there being an acceleration tube with a longitudinal center axis, a cavity, a proximal end, and a distal end, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth between the proximal-side abutment element and the distal-side abutment element, and a vibration excitation apparatus for exciting vibrations of the sonotrode being arranged in the housing, wherein the holding device comprises a vibration compensation apparatus with at least one mass and at least one spring element such that the vibration compensation apparatus makes it possible to decouple the acceleration tube from the excitation of vibrations by means of the vibration excitation apparatus. The invention also relates to a lithotripsy device for fragmenting calculi.

1. A holding device configured for a lithotripsy device and configured to fragment calculi, the holding device comprising:
a housing with a distal end and a proximal end and a sonotrode being connectable to the distal end, arranged within the housing there being an acceleration tube with a longitudinal center axis, a cavity, a proximal end, and a distal end, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus configured to generate a force to move the projectile back and/or forth between the proximal-side abutment element and the distal-side abutment element, and
a vibration excitation apparatus configured to excite vibrations of the sonotrode being arranged in the housing, wherein the holding device comprises a vibration compensation apparatus with at least one mass and at least one spring element such that the vibration compensation apparatus allows decoupling of the acceleration tube from the excitation of vibrations by the vibration excitation apparatus.

2. The holding device as claimed in claim 1, wherein, in its longitudinal direction and/or at its proximal end, the mass is arranged without a connection to the housing.

3. The holding device as claimed in claim 1, wherein the mass is directly or indirectly connected to the housing substantially transversely to the longitudinal direction of said mass by at least one connection element.

4. The holding device as claimed in claim 3, wherein the mass is directly or indirectly connected to the housing at least three radially uniformly spaced apart connection elements.

5. The holding device as claimed in claim 3, wherein a respective punctiform direct or indirect connection to the housing is formed by the at least one connection element or the connection elements.

6. The holding device as claimed in claim 3, wherein the at least one connection element or the connection elements comprises or comprise plastic.

7. The holding device as claimed in claim 1, wherein the at least one spring element is a tube portion, with a wall thickness of the tube portion being smaller than a material thickness of the mass.

8. The holding device as claimed in claim 1, wherein the vibration compensation apparatus comprises a cavity and/or a cutout to accommodate one or more of a pressure medium, and at least one sealing element.

9. The holding device as claimed in claim 1, wherein the vibration compensation apparatus is at least partially arranged around the acceleration tube.

10. The holding device as claimed in claim 1, wherein the vibration compensation apparatus is arranged concentrically around the acceleration tube.

11. The holding device as claimed in claim 1, wherein the holding device comprises a circuit board holder, the circuit board holder being at least partially arranged around the vibration compensation apparatus and the mass of the vibration compensation apparatus being connected to the circuit board holder by the at least one connection element.

12. The holding device of claim 1, wherein the holding device comprises a horn distally and a bolt proximally of the horn, the horn and the bolt surrounding a distal portion of the acceleration tube, a counter bearing is arranged on the bolt proximally of the horn and at least one piezo element as a vibration exciter is arranged and mechanically coupled between the counter bearing and the horn, the horn comprising the distal-side abutment element and/or the horn being connectable to the distal-side abutment element and/or the sonotrode and the at least one piezo element being electrically connectable to an assignable ultrasonic generator, the vibration compensation apparatus being arranged proximally on and/or of the horn, the bolt, and/or the counter bearing.

13. The holding device as claimed in claim 12, wherein the at least one spring element comprises a connection portion, the connection portion surrounding a proximal end portion of the bolt and/or being arranged proximally of the counter bearing.

14. The holding device as claimed in claim 1, wherein the mass comprises a perforation and/or, on its outer surface, at least one cutout in its longitudinal direction configured for guiding a line and/or a tube.

15. A lithotripsy device configured to fragment calculi, the lithotripsy device comprising a sonotrode and a holding device, wherein the holding device is a holding device as claimed in claim 1.
